(19) United States
(12) Patent Application Publication
(10) Pub. No.: US 2016/0122305 A1
(43) Pub. Date: May 5, 2016

(54) Topical Compositions and Methods of Use Thereof

(71) Applicant: Avon Products, Inc., Suffern, NY (US)

(72) Inventors: Kai Xi, Whitehouse Station, NJ (US); Jolanta Idkowiak-Baldys, Montebello, NY (US); Uma Santhanam, Tenafly, NJ (US); John W. Lyga, Basking Ridge, NJ (US)

(21) Appl. No.: 14/529,383

(22) Filed: Oct. 31, 2014

(51) Int. Cl. C07D 233/22; C07D 401/12; A61Q 19/02; A61K 8/49; A61Q 19/08; A61Q 19/06

(52) U.S. Cl. CPC C07D 233/22; A61Q 19/08; A61Q 19/06; A61Q 19/02; A61K 8/494; C07D 401/12

(57) ABSTRACT

The invention is generally directed to compositions useful for improving the aesthetic appearance of human skin and the use of those compounds for improving the appearance of human skin, including improving skin tone, reducing the appearance of dark circles under the eyes, and reducing inflammation in the skin. The compositions typically comprise adrenergic receptor agonists.

### FIELD OF INVENTION

The invention relates generally to topical compositions for applications to human integuments. More particularly, the invention provides topical compositions and associated methods for improving the appearance of skin, including improving skin tone, reducing dark circles around the eye area, and reducing inflammation, which can further lead to anti-aging effects, such as reduction and/or prevention of fine lines or wrinkles.

### BACKGROUND

Several skin conditions are associated with redness, inflammation and discoloration of skin. For example, rosacea, a common and chronic disorder, is characterized by flushing and persistent redness (erythema) in the central facial area. Such skin conditions may have considerable psychosocial impact and may cause embarrassment, anxiety and low self-esteem.

Although there are medications that are approved for the treatment of skin inflammation and/or redness, there exists a need for topical compositions to improve skin tone, reduce skin inflammation, reduce dark circles around eye area, and other skin indications. It is hypothesized that dilation of facial blood vessels upon various stimuli contributes to these skin conditions. Therefore, agents with vasoconstrictive activity may have symptomatic effect on these skin conditions.

Studies suggest the involvement of adrenergic receptors in the neurovascular regulation pathway. Adrenergic agonists stimulate a response from the adrenergic receptors. The five main types of adrenergic receptors are: α1, α2, β1, β2, and β3, and agonists vary in specificity between these receptors, and may be classified accordingly. There are two subclasses of α-receptor: al and α2 which are further subdivided into α1A, α1B, α1D, α2A, α2B and α2C. The β receptors are divided into β1, β2 and β3.

It is known that certain adrenergic receptor agonists possess vasoconstrictive activity and are effective to reduce erythema of rosacea. These include Brimonidine tartrate, an α1 adrenergic receptor agonist, as described in “Once-daily topical brimonidine tartrate gel 0.5% is a novel treatment of moderate to severe facial erythema of rosacea: results of two multicenter, randomized and vehicle-controlled studies,” British Journal of Dermatology, March 2012; 166(3): 633-641; Oxymetazoline, an α1 and α2 adrenergic receptor agonist, as described in “Successful Treatment of the Erythema and Flushing of Rosacea Using a Topically Applied Selective 1-Adrenergic Receptor Agonist, Oxymetazoline,” Arch Dermatol., Vol. 143 (No. 11), November 2007, and Xylometazoline, an α1A and α2A adrenergic receptor agonist, as described in “Rosacea (erythematotelangiectatic type) effectively improved by topical xylometazoline,” The Journal of Dermatology, Vol. 38, Issue 5, pp. 510-513, May 2011 However, these compounds are known to cause systemic side effects. It is therefore an object of the invention to provide compositions and methods for improvement of the appearance of skin, including, without limitation, improvement in skin tone, reduction of dark circle around eye area, and reduction of inflammation (with consequent anti-aging effects such as anti-wrinkle benefits).

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF INVENTION

In accordance with the foregoing objectives and others, the invention provides compounds, topical compositions and methods for improving the appearance of skin, including, without limitation, improvement of skin tone, reduction of dark circles around the eye area, and reduction of inflammation of human skin. Without wishing to be bound by any theory, it is believed that the compounds of the invention are adrenergic receptor (e.g., α1 and α2 adrenergic receptor) agonists, with little or no activity as β2 receptor agonists. The compounds of the invention are believed to possess vasoconstrictive activity that can lead to skin benefits, including improved skin tone, reduction of dark circle around eye area, and reduced inflammation of the skin that can lead to further anti-aging effects such as anti-wrinkle benefit for cosmetic skin care use.

One aspect of the invention provides compounds that are human adrenergic receptor agonists (in particular, at the α1 and α2 receptor, e.g., at the α1A and α2A receptor). In some embodiments, the compounds are α1A and/or α2A adrenergic receptor agonists, although it is contemplated that the compounds may act on any of α1, α2, β1, β2, and β3 adrenergic receptors or have non-specific activity (e.g., agonist activity) across two or more of these receptors. However, it will be understood that the precise mechanism of action of the compounds is not intended to limit the invention unless otherwise indicated. These compounds may effect vasoconstriction in human tissues, such as skin. The compounds of the invention are contemplated to be beneficial in improving skin appearance and reducing signs of aging (e.g., wrinkles and fine lines). The compounds are contemplated to be useful in reducing inflammation, including by topical application to the skin.

In one aspect of the invention, topical compositions are provided for improving the appearance of the human integumentary system, including improving skin tone and reducing inflammation discoloration, erythema or pigmentation of human integuments (e.g., skin) comprising, in a topically acceptable vehicle (e.g., a water-in-oil or oil-in-water emulsion, gel or an aqueous serum), an effective amount (e.g., from about 0.0001% to about 0.01%, or to about 0.1%, or to about 1%, or to about 10% by weight) of a compound (e.g., an adrenergic receptor agonist compound) according to formula (I):

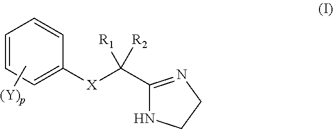

wherein X may be selected from oxygen, sulfur, NR*, or CR*R* (but is typically oxygen);

R₁ and R₂ are independently selected from hydrogen or R* (typically at least one of R₁ and R₂ is R*), wherein R₁ and R₂ may together form a 3-6-membered ring; and wherein R₁ is typically lower alkyl (e.g., methyl, ethyl, propyl, butyl, etc.) and more typically methyl; and R₂ is typically hydrogen;

Y is selected independently at each occurrence from hydrogen, —F; —Cl; —Br; —I; —OH, —OR*; —NH₂; —NHR*; —N(R*)₂⁺; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof;

p is an integer from 1-5, indicating the number of available positions on the phenyl ring having a group Y attached thereto; for example, p may be one, two, three or four with Y being the same or different at each position; and in some embodiments, p is one; the groups Y may be attached ortho, meta and/or para to the group X; and in one embodiment, there is a group Y ortho to X; in one embodiment p is one and Y is ortho to X;

R* is independently at each occurrence hydrogen or a straight chained, branched, or cyclic C₁-C₂₀ (or C₁₋₁₆ or C₁₋₁₂ or C₁₋₈ or C₁₋₆ or C₁₋₄) hydrocarbon radical, such as alkyl, alkenyl, alkynyl, aryl, aryl-alkyl, alkyl-aryl, heteroaryl, etc., optionally substituted with one or more groups Y or with 1-6 heteroatoms selected from nitrogen, oxygen, sulfur, or halogen and combinations thereof; and ins some embodiments, R* is methyl; and wherein any two adjacent R* moieties may together form a 3-6 membered ring;

Topically acceptable salts (e.g., acid addition salts) of the compounds according to formula (I) are also suitable.

In another implementation, topical compositions are provided comprising, in a topically acceptable vehicle (e.g., an emulsion, gel or serum), an effective amount (e.g., from about 0.0001% to about 0.01%, or to about 0.1%, or to about 1%, or to about 10% by weight) of a compound (e.g., an adrenergic receptor agonist compound) having the structure of formula (II), or a topically acceptable salt thereof:

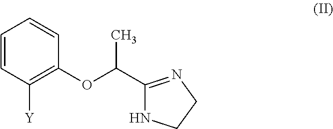

In the compounds of Formula (II) Y is selected from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof; and in some embodiments, Y comprises a five or six-membered ring, optionally aromatic and optionally containing one, two or three heteroatoms (e.g., oxygen, sulfur, and/or nitrogen) in the ring.

In another implementation, topical compositions are provided comprising, in a topically acceptable vehicle (e.g., an emulsion, gel, or serum), an effective amount (e.g., from about 0.0001% to about 0.01%, or to about 0.1%, or to about 1%, or to about 10% by weight) of a compound (e.g., an adrenergic receptor agonist compound) selected from

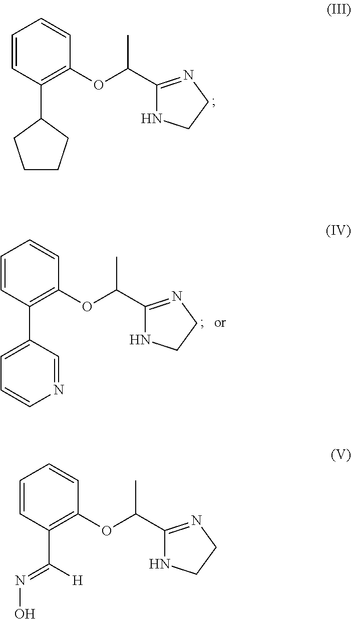 [SMILES: CC(Oc1ccccc1C1CCCC1)C1=NCCN1 ; CC(Oc1ccccc1-c1cccnc1)C1=NCCN1 ; CC(Oc1ccccc1/C=N/O)C1=NCCN1]

and topically acceptable salts thereof. Compounds III, IV, and V may be provided as racemic mixtures, or as pure R or S enantiomers, or as mixtures of R and S enantiomers having an enantiomeric excess of one or the other of R or S of more than 60% or more than 70% or more than 80% or more than 90%.

Topically acceptable salts (e.g., acid addition salts) of the compounds of formulas (I) or (II) are also suitable. The compositions of the invention typically comprise (e.g., from about 50% to about 99% by weight) a vehicle, for example a water-in-oil or oil-in-water emulsion, and may further include various adjuvants such as thickeners, rheology modifiers, emulsifiers, gellants, emollients, humectants, UV absorbers, antioxidants, pH adjusters, chelators, film formers, waxes, preservatives, colorants, fragrances, and the like. The adjuvants may comprise, individually or collectively, from about 0.00001% to about 98% by weight of the composition (more typically from about 0.001-50% or 0.001-20%). The topical preparations of the invention may further include one or more additional active agents, such as a retinoid (e.g., retinol, retinyl palmitate, retinyl acetate, retinaldehyde, retinoic acid, etc.), an antioxidant (e.g., ascorbic acid, BHT, thiodipropionic acid or esters thereof, including dilauryl thiodipropionate), α-hydroxy acids (e.g., glycolic acid, lactic acid, etc.), collagenase inhibitors, anti-inflammatories, anti-acne agents, salicylic acid and derivatives (e.g., C₁₋₁₆ esters), depigmenting agents (e.g., hydroquinone, kojic acid, etc.), N-acetyl tyrosinamide, and botanicals, to name a few. In one embodiment, the compositions include one or more of hydroquinone, kojic acid, salicylic acid or its derivatives, thiodipropionic acid (or C₁₋₂₀ mono- or diesters thereof), and retinoids (e.g., retinol). Additional actives may individually or collectively comprise from about 0.0001% to about 20% by weight of the composition.

Topical compositions comprising any of the compounds of formulas (I-V) may be applied topically to skin for a time sufficient to achieve an improvement in skin appearance, including, without limitation, improved skin tone and/or reduction of the appearance of dark circles under the eyes and/or reduction in inflammation or erythema in the area of application. This may entail application at least once, twice or more daily for at least one week, at least two weeks, at least four weeks, or at least eight weeks or longer. In some embodiments, the compositions are topically applied daily (at least once) to achieve and/or maintain improved skin tone and/or reduction of dark circles and/or inflammation or erythema in the skin.

In one aspect of the invention, a method for modulation of adrenergic receptors (e.g., in human skin) is provided comprising administering (e.g., topical application) the compounds of formulas (I-V) to a human (e.g., to human skin). Without wishing to be bound by any particular theory, it is believed that the compounds of formulas (I-V) act as adrenergic receptor (e.g., al and α2 receptor, or the α1A and α2A receptor) agonists i.e., adrenergic receptor activators, which may result in vasoconstriction, which may lead to improvements in the aesthetic appearance of skin, including improved skin tone, reduction of dark circles around the eye area, and reduced inflammation, which may further lead to anti-aging effects, such as reduction, and/or prevention of fine lines or wrinkles and other aesthetic effects. Associated methods of achieving vasoconstriction are also provided.

These and other aspects of the present invention will be better understood by reference to the following detailed description and appended claims.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

All percentages given herein refer to the weight percentages of a particular component relative to the entire composition, including the vehicle, unless otherwise indicated. It will be understood that the sum of all weight % of individual components within a composition will not exceed 100%.

Unless otherwise provided all hydrocarbon moieties described herein (e.g., alkyl, alkenyl, alkynyl, aryl, alkyl-aryl, aryl-alkyl, heteroalkyl, etc.) will comprise from 1-32 atoms in the chain and/or ring. Examples include C₁₋₂₆, C₁₋₂₀, C₁₋₁₈, C₁₋₁₆, C₁₋₁₂, C₁₋₈, C₁₋₆, and C₁₋₄ hydrocarbons.

Compounds of the invention may be provided as racemic mixtures, or as pure R or S enantiomers, or as mixtures of R and S enantiomers having an enantiomeric excess of one or the other, for example and enantiomeric excess (of R or S) of more than 60% or more than 70% or more than 80% or more than 90%.

All terms used herein are intended to have their ordinary meaning unless otherwise provided. The phrases “cosmetically acceptable,” “topically acceptable” and “dermatologically acceptable” are used interchangeably and are intended to mean that a particular component is generally regarding as safe and non-toxic for application to a human integument (e.g., skin) at the levels employed. The term “prevent,” as used herein, includes delaying the onset of or progression of a particular sign of skin aging. The term “treat” includes reducing, diminishing the appearance or severity of, eliminating, ameliorating, forestalling, slowing the progression of, and/or delaying the onset of a given skin indication. The phrase “individual in need thereof” refers to a human that could benefit from improved dermal appearance or health, including males or females. In some embodiments, an individual in need thereof has a clinical diagnosis of a particular skin indication. In some embodiments, the individual in need thereof is a female (e.g., a pre- or post-menopausal female). The term “skin” includes, without limitation, the lips, skin of the face, hands, arms, neck, scalp, and chest.

As used herein, the term “consisting essentially of” is intended to limit the invention to the specified materials or steps and those that do not materially affect the basic and novel characteristics of the claimed invention, as understood from a reading of this specification. This may include, without limitation, specificity to adrenergic receptor subtype(s).

As used herein, the term “modulator” may refer to an agonist or an antagonist. In some embodiments, the compounds are adrenergic receptor agonists.

As used herein, a compound is said to have activity against a receptor subtype if it has an EC50 value of less than about 1,000 nm, preferably less than about 500 nm, more preferably less than about 400 nm, even more preferably less than about 300 nm, or about 250 nm, or about 200 nm. In some embodiments, an agonist will have an EC50 of less than 100 nm, less than 50 nm, less than 10 nm, or less than 1 nm. A compound is said to have little or no activity against a receptor subtype if it has an EC50 value of greater than about 1,000 nm, or greater than about 1,500 nm, or greater than a about 2,000 nm, or greater than about 2,500 nm.

The compositions may be used for, among other things, improving the appearance of human skin, including, without limitation, improving skin tone, reducing dark circles under the eye area, and/or reducing inflammation of the human integumentary system, including but not limited to, keratinous surfaces such as skin, hair, lips, and nails. The compositions are typically topical compositions that once applied to the integumentary system result in the narrowing of the blood vessels (vasoconstriction) of the integument to which it is applied.

The compounds of the invention (e.g., adrenergic receptor agonist compounds) may have the structure of formula (I):

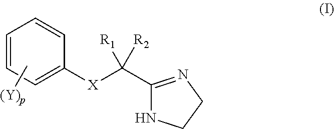

where X may be selected from oxygen, sulfur, NR*, or CR*R*; and where X is typically oxygen;

R₁ and R₂ are independently selected from hydrogen or R*, and wherein R₁ and R₂ may together form a 3-6-membered ring; and in some embodiments, R₁ and/or R₂ are methyl, ethyl, propyl, or butyl, and in other embodiments R₁ is methyl; and in other embodiments R₂ is hydrogen;

Y is selected independently at each occurrence from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical; and combinations thereof;

p is an integer from 1-5, indicating the number of available positions on the phenyl ring having a group Y attached thereto; for example, p may be one, two, three or four with Y being the same or different at each position; and in some embodiments, p is one; the groups Y may be attached ortho, meta and/or para to the group X; and in one embodiment, there is a group Y ortho to X; in one embodiment p is one and Y is ortho to X;

R* is independently at each occurrence hydrogen or a straight chained, branched, or cyclic C₁-C₂₀ (or C₁₋₁₆ or C₁₋₁₂ or C₁₋₈ or C₁₋₆ or C₁₋₄) hydrocarbon radical, such as alkyl, alkenyl, alkynyl, aryl, aryl-alkyl, alkyl-aryl, heteroaryl, etc., optionally substituted with one or more groups Y or with 1-6 heteroatoms selected from nitrogen, oxygen, sulfur, or halogen and combinations thereof; and ins some embodiments, R* is methyl; and wherein any two adjacent R* moieties may together form a 3-6 membered ring;

and topically acceptable salts thereof.

In some embodiments, X is oxygen.

In some embodiments, R₁ and R₂ may independently be selected from hydrogen, methyl, ethyl, propyl, butyl, pentyl, and hexyl. In certain implementations, R₁ is methyl. In some embodiments, R₂ is hydrogen. In some embodiments, Y is present at one, two, three, four or five positions on the ring to which it is attached. In some embodiments, Y is present at one position on the ring to which it is attached. In some embodiments, Y is an aliphatic C₁-C₈ hydrocarbon radical; a C₅-C₈ aromatic hydrocarbon radical; or a C₅-C₈ heteroaryl radical; or a group —CH(═N—OH).

In another implementation, the compound of formula (I) will have the structure of formula (II):

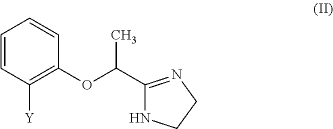

In the compounds of Formula (II) Y is selected from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof; and in some embodiments, Y comprises a five or six-membered ring, optionally aromatic and optionally containing one, two or three heteroatoms (e.g., oxygen, sulfur, and/or nitrogen) in the ring.

In other implementation, the compound of formula (I) or (II) will have the structure selected from formulas (I)-(III):

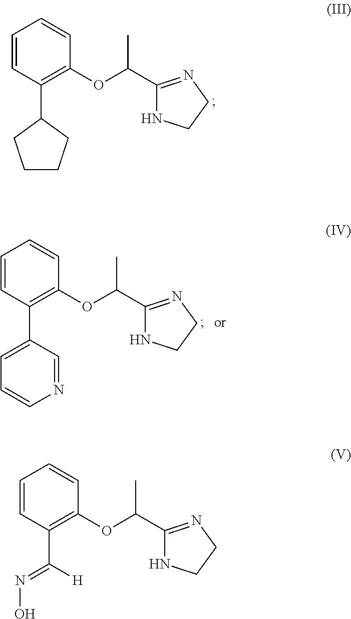 [SMILES: CC(Oc1ccccc1C1CCCC1)C1=NCCN1 ; CC(Oc1ccccc1-c1cccnc1)C1=NCCN1 ; CC(Oc1ccccc1/C=N/O)C1=NCCN1]

and topically acceptable salts thereof. Compounds III, IV, and V may be provided as racemic mixtures, or as pure R or S enantiomers, or as mixtures of R and S enantiomers having an enantiomeric excess of one or the other of more than 60% or more than 70% or more than 80% or more than 90%.

The compositions according to the invention can be formulated in a variety of forms for topical application and will typically comprise from about 0.00001% by weight to about 20% by weight of one or more compounds according to Formula (I)-(V), and preferably will comprise from about 0.0001% by weight to about 10% by weight, and more preferably from about 0.001% by weight to about 5% by weight of the composition. In one embodiment, the active will comprise from about 0.01% by weight to about 0.1% by weight or to about 0.5% by weight or to about 1% by weight of the composition. The compositions will comprise and effective amount of the compound according to Formula (I)-(V), by which is meant an amount sufficient to have a vasoconstrictive effect in a given area of skin when topically applied thereto.

The cosmetically acceptable vehicle may be in the form of an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. As used herein, the term “oil” includes silicone oils unless otherwise indicated. The emulsion may include an emulsifier, such as a nonionic, anionic or amphoteric surfactant, or a gellant, typically in an amount from about 0.001% to about 10% by weight. The gellant may be an oil phase or aqueous phase.

The cosmetically acceptable vehicle may include water; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane (IDD) and isohexadecane; silicone oils such as cyclomethicone, dimethicone, dimethicone cross-polymer, polysiloxanes and their derivatives, preferably organomodified derivatives including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous vehicles may include one or more solvents miscible with water, including lower alcohols, such as ethanol, isopropanol, and the like. The vehicle may comprise from about 50% to about 99% by weight of the composition.

In one embodiment of the invention, the compositions may include additional skin actives, including but not limited to, retinoids, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, α-hydroxy acids, and advanced glycation end-product (AGE) inhibitors, to name but a few. The amounts of these various ingredients are those conventionally used in the cosmetic field to achieve their intended purpose, and range individually or collectively typically from about 0.001 wt % to about 20 wt % by weight of the composition. The nature of these ingredients and their amounts must be compatible with the production and function of the compositions of the disclosure.

Exemplary anti-aging components include, without limitation, botanicals (e.g., Butea frondosa extract); phytol; phytonic acid; retinoids; hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.), to name a few.

Exemplary retinoids include, without limitation, retinoic acid (e.g., all-trans, or 9-cis, or 13-cis), and derivatives thereof, retinaldehyde, retinol (Vitamin A) and esters thereof, such as retinyl palmitate, retinyl acetate and retinyl propionate, and salts thereof. Particular mention may be made of retinol. When present, the retinoids will typically be included in amounts from about 0.0001% to about 5% by weight, more typically from about 0.01% to about 2.5% by weight, or from about 0.1% to about 1.0% by weight. Compositions according to this embodiment will typically include an antioxidant such as ascorbic acid and/or BHT and/or a chelating agent such as EDTA or a salt thereof (e.g., disodium EDTA).

In some embodiments, the compositions of the invention may further comprise one or more of Amorphophallus Campanulatus Rhizome/Root Extract, Glycine Max (Soybean) Symbiosome Extract, Vitis Vinifera (Grape) Fruit Cell Extract, Malus Domestica Fruit Cell Culture Extract, Eclipta Prostrata Extract, Palmitoyl Lysyl Aminovaleroyl Lysine, Palmitoyl Oligopeptide, Palmitoyl Tetrapeptide-10, Palmitoyl Tetrapeptide-7, Melicope Leaf Extract, Saccharomyces Ferment Lysate Filtrate, Thiazolylalanine, Mesyloxybenzyl Isobutylbenzenesulfonamide, Phytol, Thiodipropionic Acid, Dilauryl Thiodipropionate, Coffea Arabica (Coffee) Seed Oil, Foeniculum Vulgare (Fennel) Fruit Extract, Ceramide 2, Crataegus Monogyna Fruit Extract, Tocopherol, Prunus Amygdalus Dulcis (Sweet Almond) Seed Extract, Hydrolyzed Hibiscus Esculentus Extract, Cocos Nucifera (Coconut) Fruit Juice, Punica Granatum Fruit Juice, Portulaca Oleracea Extract, Olea Europaea (Olive) Leaf Extract, Saxifraga Sarmentosa Extract, Daucus Carota Sativa (Carrot) Root Extract, Passiflora Incarnata Flower Extract, Morus Nigra Root Extract, N-Hydroxysuccinimide, Scutellaria Baicalensis Root Extract, Foeniculum Vulgare (Fennel) Fruit Extract, Medicago Sativa (Alfalfa) Extract, Biotin, Peg-10 Rapeseed Sterol, cis-6-nonenol, Arginine, Aronia Melanocarpa Fruit Juice, Zizyphus Jujuba Fruit Extract, Petasites Hybridus Leaf Extract, Saccharomyces/Platinum Ferment, Glycine Soja (Soybean) Seed Extract, Phytol, Helianthus Annuus (Sunflower) Seed Extract, Andrographolide, Bisabolol, Panthenol, Hydrolyzed Milk Protein, Adenosine Cyclic Phosphate, Amethyst Extract, Saccharomyces/Calcium Ferment, Saccharomyces/Copper Ferment, Saccharomyces/Potassium Ferment, Saccharomyces/Magnesium Ferment, Saccharomyces/Zinc Ferment, Saccharomyces/Manganese Ferment, Saccharomyces/Iron Ferment, Saccharomyces/Silicon Ferment, and Saccharomyces Ferment Lysate Filtrate. These additional active(s) may be present individually or collectively in an amount from about 0.0001% to about 20% by weight of the composition.

In another embodiment, the topical compositions of the present invention may also include one or more of the following: a skin penetration enhancer; an emollient, such as isopropyl myristate, petrolatum, volatile or non-volatile silicones oils (e.g., methicone, dimethicone), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; a sunscreen, such as avobenzone or octyl methoxycinnamate; an exfoliating agent; and an antioxidant.

Suitable exfoliating agents include, for example, alpha-hydroxy acids, beta-hydroxy acids, oxa-acids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.001% to about 20% by weight of the composition.

Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives, including tocopheryl acetate; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Antioxidants may comprise, individually or collectively, from about 0.001% to about 10% (w/w), or from about 0.01% to about 5% (w/w) of the total weight of the composition.

Other additives include: vitamins, such as tocopherol and ascorbic acid; vitamin derivatives such as ascorbyl monopalmitate, tocopheryl acetate, and Vitamin E palmitate; thickeners such as hydroxyalkyl cellulose, carboxymethylcellulose, carbombers, and vegetable gums such as xanthan gum; gelling agents, such as ester-terminated polyester amides; structuring agents; metal chelating agents such as EDTA or salts thereof; pigments; colorants; and pH adjusters (citric acid, ethanolamine, sodium hydroxide, etc.). The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders.

In addition, the compositions contemplated by this disclosure can include one or more compatible cosmetically acceptable adjuvants commonly used and known by the skilled practitioner, such as colorants, pearls, chromalites, micas, pigments, dyes, fragrances, emollients, humectants, preservatives, vitamins, chelators, thickeners, anesthetics, anti-allergenics, antifungals, antimicrobials, other anti-inflammatory agents, antioxidants, antiseptics, depigmenting agents, film formers, insect repellents, pharmaceutical agents, photostabilizing agents, sunscreens, stabilizers, surfactants, thickeners, viscosity modifiers, and botanicals. The topical compositions of the present disclosure may also include a skin penetration enhancer, a surface smoother, a skin plumper, an optical diffuser, an exfoliation promoter, and an antioxidant. Details with respect to these and other suitable cosmetic ingredients can be found in the “International Cosmetic Ingredient Dictionary and Handbook,” 10th Edition (2004), published by the Cosmetic, Toiletry, and Fragrance Association (CTFA), at pp. 2177-2299, which is herein incorporated by reference in its entirety. The amounts of these various substances are those that are conventionally used in the cosmetic or pharmaceutical fields, for example, they can constitute from about 0.01% to about 20% of the total weight of the composition.

A sunscreen may be included to protect the skin from damaging ultraviolet rays. In an illustrative embodiment of the present disclosure, the sunscreen provides both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

In one embodiment, the topical composition will have a pH range from 1 to 13, with a pH in the range of from 2 to 12 being typical. In some embodiment, the composition will have a pH in the range of from 3.5 to 7 or from 7-10.5. In some embodiments, the pH will be in the range of 3-4, or 4-5, or 5-6, or 6-7, or 7-8, or 8-9, or 9-10, or 10-11, or 11-12. Suitable pH adjusters such as sodium hydroxide, citric acid and triethanolamine may be added to bring the pH within the desired range.

Another embodiment of the present disclosure is directed to the delivery of the described compositions by the use of targeted delivery systems, for example, liposomes, microspheres (see, e.g., U.S. Pat. No. 5,770,222 to Unger et al.), and the like, so that the components and/or active constituents can more readily reach and affect the subcutaneous layer of the area of application, e.g., face or neck, or the other area of the skin.

The compositions may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration. Preferably the composition is formulated as a lotion, cream, ointment, or gel.

In some embodiments, the compositions and methods are for improving skin tone (in particular for skin of the face), which includes evening, lightening, or optimizing the tone of the skin. In other embodiments, the compositions and methods are for treating dark circles under the subject's eye area. In other embodiments, the compositions and methods are for reducing redness and inflammation in the skin, including without limitation treating, ameliorating, diminishing the appearance of, or preventing inflammation in the skin. The compositions may be applied directly to a localized site of affected skin.

The compositions and methods of the invention are also contemplated to be useful for treating, reducing, ameliorating, improving, alleviating, and/or eliminating the dermatological effects of aging and/or environmental stress. The compositions and methods are suitable for use in treating dermatological conditions of the skin, including treatment of fine lines and wrinkles, reducing hyperpigmentation or unwanted pigmentation, or treatment of erythema in numerous areas of the body, including, without limitation, the face, forehead, lips, neck, arms, hands, legs, knees, feet, chest, back, groin, buttocks, thighs, and the like. In one embodiment, the compositions are applied topically to the skin of the face.

In another aspect of the invention, the compositions are applied topically to improve the aesthetic appearance of human skin. The method comprises topically applying to an area of the skin in need thereof a composition comprising an effective amount of a compound of Formula (I) or (II) for a time sufficient to improve the aesthetic appearance of said human skin. The compositions are topically applied to the skin in effective amounts, by which is meant an amount sufficient to achieve a measurable improvement in skin health or reduction in one or more dermatological signs of aging with daily (once, twice, etc.) administration, typically for a period of at least one week or more.

The aesthetic improvement of human skin may be an improvement of any attribute or characteristic of skin, including without limitation:

(a) treatment, reduction, and/or prevention of fine lines or wrinkles;

(b) reduction of skin pore size;

(c) improvement in skin thickness, plumpness, and/or tautness;

(d) improvement in skin smoothness, suppleness and/or softness;

(e) improvement in skin tone, radiance, and/or clarity;

(f) improvement in procollagen, and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization;

(o) increase in skin elasticity and/or resiliency;

(p) treatment, reduction, and/or prevention of skin sagging;

(q) improvement in skin firmness; and

(r) reduction of pigment spots and/or mottled skin; and

(s) improvement of optical properties of skin by light diffraction or reflection.

In a related implementation, a method is provided for the treatment of wrinkles and/or fine lines on the skin human skin (typically, skin of the face) comprising topically applying to an area of the skin in need thereof (e.g., applying to a wrinkle or fine line) a composition of Formula (I)-(V), for a time sufficient to improve the aesthetic appearance of said human skin. The treatment may be a least once or twice daily and may last for a period of at least four weeks, typically at least eight weeks or longer. The composition may optionally further comprise a retinoid (e.g., retinol or retinyl palmitate) and/or an alpha-hydroxy acid (e.g., glycolic acid) and/or a beta-hydroxy acid (e.g., salicylic acid or derivative) in amounts effective to improve the appearance of skin.

In a related aspect, methods are provided for enhancing the production of collagen or procollagen in human skin comprising topically applying to an area of the skin in need thereof (e.g., sagging skin, thinning skin, skin suffering from wrinkles and fine lines, etc.) a topical composition comprising a cosmetically acceptable vehicle, and an effective amount of a compound of Formula (I)-(V), for a time sufficient to improve the appearance thereof. The treatment may be a least once or twice daily and may last for a period of at least four weeks, typically at least eight weeks or longer. The composition may optionally further comprise a retinoid and/or an alpha-hydroxy acid (e.g., glycolic acid) and/or a beta-hydroxy acid (e.g., salicylic acid or a derivative) in amounts effective to improve the appearance of skin.

In yet another aspect of the invention, methods are provided for reducing the severity of, reducing the number of, or preventing or forestalling the onset of, wrinkles or fine lines on human skin comprising topically applying to an area of the skin in need thereof (e.g., wrinkled skin), an effective amount (e.g., 0.0001%-1% by weight, w/w) of a compound of Formula (I)-(V), in combination with an effective amount (e.g., 0.01%-5% by weight, w/w) of retinol and/or an effective amount (e.g., 0.001%-5% by weight, w/w) of an alpha-hydroxy acid (e.g., glycolic acid) and/or a beta-hydroxy acid (e.g., salicylic acid).

The invention provides a method for treating aging skin by topically applying a composition comprising a collagen-stimulating compound of Formula (I)-(V), typically in a cosmetically acceptable vehicle, over the affected area for a period of time sufficient to remediate, reverse, reduce, ameliorate, or prevent dermatological signs of aging. Generally, the improvement in the condition and/or aesthetic appearance is selected from the group consisting of: reducing dermatological signs of chronological aging, photo-aging, hormonal aging, and/or actinic aging; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; improving the appearance of suborbital lines and/or periorbital lines; reducing the appearance of crow's feet; rejuvenating and/or revitalizing skin, particularly aging skin; reducing skin fragility; preventing and/or reversing of loss of glycosaminoglycans and/or collagen; ameliorating the effects of estrogen imbalance; preventing skin atrophy; preventing, reducing, and/or treating hyperpigmentation or hypopigmentation; minimizing skin discoloration; improving skin tone, radiance, clarity and/or tautness; preventing, reducing, and/or ameliorating skin sagging; improving skin firmness, plumpness, suppleness and/or softness; improving procollagen and/or collagen production; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause; improving communication among skin cells; increasing cell proliferation and/or multiplication; increasing skin cell metabolism decreased by aging and/or menopause; retarding cellular aging; improving skin moisturization; enhancing skin thickness; slowing or halting skin thinning; increasing skin elasticity and/or resiliency; enhancing exfoliation; improving microcirculation; decreasing and/or preventing cellulite formation; and any combinations thereof. In some embodiments, each of the forgoing is associated with female skin.

In some embodiments, the compounds of Formulas (I)-(V) will be used to reduce the severity of fine lines or wrinkles, often in combination with retinol. The composition will typically be applied to the skin one, two, or three times daily for as long as is necessary to achieve desired results. The treatment regimen may comprise daily application for at least one week, at least two weeks, at least four weeks, at least eight weeks, or at least twelve weeks or more. Chronic treatment regimens are also contemplated. The effect of a composition on the formation or appearance of fine lines and wrinkles can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin). In one embodiment, the cosmetic compositions of the invention will be applied to the skin in an amount from about 0.001 to about 100 mg/cm², more typically from about 0.01 to about 20 mg/cm², or from about 0.1 to about 10 mg/cm².

It is also contemplated that the compositions of the invention will be useful for treating thin skin by topically applying the composition to thin skin of an individual in need thereof. “Thin skin” is intended to include skin that is thinned due to chronological aging, menopause, or photo-damage and skin that is thinning prematurely. In some embodiments, the treatment is for thin skin in men, whereas other embodiments treat thin skin in women, pre-menopausal or post-menopausal, as it is believed that skin thins differently with age in men and women, and in particular in women at different stages of life.

The method of the invention may be employed prophylactically to forestall aging including in individuals that have not manifested signs of skin aging, most commonly in individuals under 25 years of age. The method may also reverse or treat signs of aging once manifested as is common in individuals over 25 years of age, or to slow the progression of dermatological aging in such individuals.

In one embodiment, the compositions of the invention are applied to human skin to reduce sebum production or improve the appearance of skin affected by cellulite, and/or reduce unwanted lipogenesis or increase lipolysis. In this embodiment, the compounds of Formulas (I)-(V) can be formulated in cosmetically acceptable vehicles (as described herein) and may include one or more additional agents such as anti-acne ingredients (e.g., salicylic acid, benzoyl peroxide and other peroxides, sulfur, retinoids, etc.) in the case of a facial composition, or, in the case of a cellulite treatment, the formulation may comprise any ingredients suitable for treatment of cellulite, including without limitation, perilla oil and other unsaturated fatty oils and omega-3 fatty acids such as alpha-linolenic acid; caffeine; theophylline; xanthines; retinoids (e.g., retinol); and the like. A cellulite treatment according to the invention will typically be applied topically to skin suffering from cellulite, including skin of the buttocks and thighs for a period of time sufficient to improve the appearance thereof, including for example, daily treatment for at least four weeks, at least eight weeks, at least twelve weeks, or longer. In one embodiment, the compositions are topically applied to treat acne.

In another embodiment, the compounds of Formulas (I)-(V) are intended for oral use, including for pharmaceutical use. Pharmaceutical formulations will include pharmaceutically acceptable carriers (i.e., diluents and excipients). The pharmaceutical compositions may be included in solid dosage forms, including compressed tablets and capsules, or in liquid or powder forms. Pharmaceutical dosage forms will typically include from about 0.5 mg to about 200 mg, or from about 1 mg to about 100 mg of the compound of Formulas (I)-(V). The dosage forms may be immediate release, in which case they will typically comprise a water-soluble or dispersible carrier such as microcrystalline cellulose, mannitol, hydroxypropyl methyl cellulose, PVP or the like, or may be delayed, sustained, or modified release, in which case they may comprise water-insoluble polymers such as cellulose ethers (e.g., ethylcellulose), alone or in combination with water soluble or dispersible polymers, to regulate the rate of dissolution of the dosage form in the stomach.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### EXAMPLES

The following example illustrates a specific aspect of the instant description. The example should not be construed as limiting, as the example merely provides specific understanding and practice of the embodiments and its various aspects.

### Example 1

The Compounds of Formulas (III)-(V) were subjected to an in vitro screening against α1A, α2A, and β2 adrenergic receptors using SelectScreen® Cell-based GPCR Profiling Service from Life Technologies. In each of the three agonist assays (α1A, α2A, and β2), cells were thawed and resuspended in Assay Media (DMEM, 10% dialyzed FBS, 25 mM HEPES pH 7.3, 0.1 mM NEAA, 100 U/mL/100 μg/mL Pen/Strep) to a concentration of 312,500 cells/mL. 32 μL of cell suspension (10,000 cells) were added to each well of a 384-well TC-Treated assay plate. Cells in Assay Media were incubated for 16-24 hours in the plate at 37° C./5% CO₂ in a humidified incubator. 4 μL of a 10× serial dilution of a control agonist (phenylephrine for α1A; UK 14304 for α2A; isoproterenol for β2) or a Compound III-V were added to appropriate wells of the plate. 4 μL of Assay Media was added to all wells to bring the final assay volume to 40 μL. The plate was incubated for 5 hours at 37° C./5% CO₂ in a humidified incubator. 8 μL of 1 μM Substrate+Loading Solution was added to each well and the plate was incubated for 2 hours at room temperature. The plate was read on a fluorescence plate reader.

Results are summarized in Table 1.

TABLE 1

| | α1A | | α2A | | β2 | |
|---|---|---|---|---|---|---|
| | Activity, | | Activity, | | Activity, | |
| Compound | EC₅₀ | | EC₅₀ | | EC₅₀ | |
| 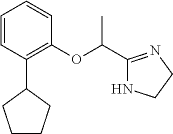 [SMILES: CC(Oc1ccccc1C1CCCC1)C1=NCCN1] (III) | <0.119 | nM | 18.7 | nM | >6300 | nM |
| 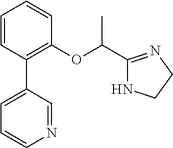 [SMILES: CC(Oc1ccccc1-c1cccnc1)C1=NCCN1] (IV) | 13 | nM | >25000 | nM | >25000 | nM |
| 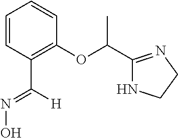 [SMILES: CC(Oc1ccccc1/C=N/O)C1=NCCN1] (V) | 32.6 | nM | 277 | nM | >63000 | nM |

As shown in Table 1, the compounds of Formulas (III)-(V) stimulate α1A adrenergic receptor activity and/or α2A receptor activity, but are not specifically active at the β2 receptor. As used herein, a compound has insignificant activity against the β2 receptor if the EC₅₀ is greater than about 500 nM. In some embodiments, the adrenergic receptor agonists will have an EC₅₀ at the β2 receptor of greater than 1,000 nM or greater than 1,500 nM, or greater than 2,000 nM. A compound is a “selective” agonist if it has activity at α1A and/or α2A receptors with an EC₅₀ of less than 1,000 nM (preferably less than 500 nM) and insignificant activity at the β2 receptor. In other words, Compound (III) is a selective α1A and α2A receptor agonist (e.g., insignificant activity against β2), Compound (IV) is a selective α1A receptor agonist, and Compound (V) is a selective α1A and α2A receptor agonist.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

### EMBODIMENTS

1. A topical composition comprising, in a topically acceptable vehicle, an effective amount of a compound capable of modulation of adrenergic receptors having the structure of formula (I):

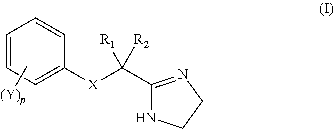

wherein X is selected from oxygen, sulfur, NR*, or CR*R*;
R₁ and R₂ are independently selected from hydrogen or R*, wherein R₁ and R₂ may together form a 3-6-membered ring;
Y is selected independently at each occurrence from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof;
p is an integer from 1-5;
R* is independently at each occurrence hydrogen or a straight chained, branched, or cyclic C₁-C₂₀ hydrocarbon radical, optionally substituted with one or more groups Y or with 1-6 heteroatoms selected from nitrogen, oxygen, sulfur, or halogen and combinations thereof;
and topically acceptable salts thereof.
2. The topical composition of claim 1, wherein X is oxygen.
3. The topical composition of claim 1, wherein R₁ is methyl.
4. The topical composition of claim 1, wherein the compound has the structure of formula (II):

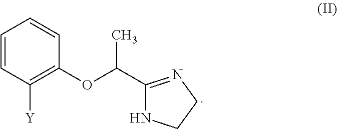

5. The topical composition of claim 1, wherein the compound is selected from the group consisting of:

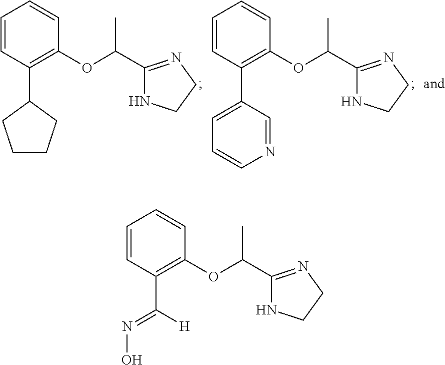

and topically acceptable salts thereof.
6. The topical composition according to claim 5, wherein the compound has the structure:

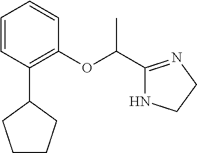 [SMILES: CC(Oc1ccccc1C1CCCC1)C1=NCCN1]

and topically acceptable salts thereof.
7. The topical composition according to claim 5, wherein the compound has the structure:

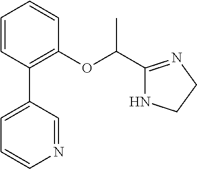 [SMILES: CC(Oc1ccccc1-c1cccnc1)C1=NCCN1]

and topically acceptable salts thereof.
8. The topical composition according to claim 5, wherein the compound has the structure:

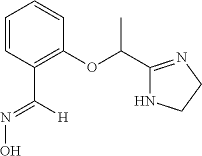 [SMILES: CC(Oc1ccccc1/C=N/O)C1=NCCN1]

and topically acceptable salts thereof.
9. The topical composition of claim 1, wherein the compound of formula (I) is capable of modulating adrenergic receptors of subtype α1A, α2A, and/or β2.
10. The topical composition of claim 9, wherein said compound is an adrenergic receptor agonist.
11. The topical composition of claim 9, wherein said compound is an agonist of α1A and/or α2A adrenergic receptors.
12. The topical composition of claim 9, wherein said compound has insubstantial agonist activity against β2 adrenergic receptor.
13. The composition according to claim 1, wherein said cosmetically acceptable vehicle comprises a water-in-oil, oil-in-water, silicone-in-water, or water-in-silicone emulsion and further comprises an emulsifier.
14. A method for modulating adrenergic receptors in human skin comprising topically applying to an area of the skin in need thereof a composition comprising, in a cosmetically acceptable vehicle, an effective amount of a compound having the structure of formula (I):

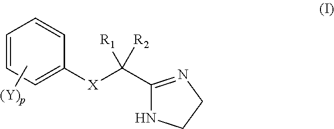

wherein X is selected from oxygen, sulfur, NR*, or CR*R*;
R₁ and R₂ are independently selected from hydrogen or R*, wherein R₁ and R₂ may together form a 3-6-membered ring;
Y is selected independently at each occurrence from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof;
p is an integer from 1-5;
R* is independently at each occurrence hydrogen or a straight chained, branched, or cyclic C₁-C₂₀ hydrocarbon radical, optionally substituted with one or more groups Y or with 1-6 heteroatoms selected from nitrogen, oxygen, sulfur, or halogen and combinations thereof;
and topically acceptable salts thereof.
15. The method according to claim 14, wherein X is oxygen.
16. The method according to claim 14, wherein R₁ is methyl.
17. The method according to claim 14, wherein the compound has the structure of formula (II):

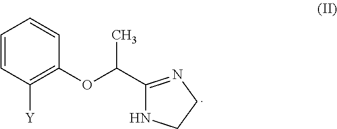

18. The method according to claim 14, wherein the compound of formula (I) is capable of modulating adrenergic receptors of subtype α1A, α2A, and/or 132.
19. The method according to claim 14, wherein the compound is selected from the group consisting of:

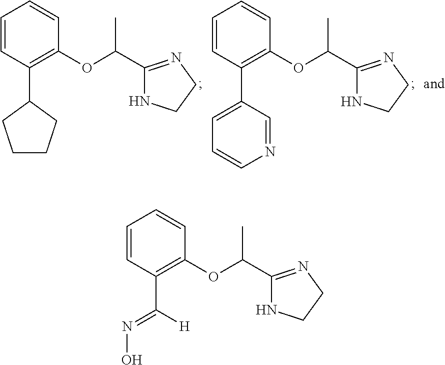

and topically acceptable salts thereof.

20. The method according to claim 19, wherein the compound has the structure:

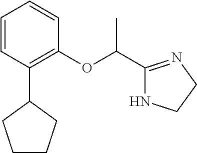 [SMILES: CC(Oc1ccccc1C1CCCC1)C1=NCCN1]

and topically acceptable salts thereof.
21. The method according to claim 19, wherein the compound has the structure:

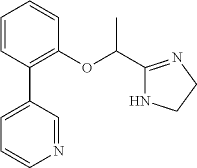 [SMILES: CC(Oc1ccccc1-c1cccnc1)C1=NCCN1]

and topically acceptable salts thereof.
22. The method according to claim 19, wherein the compound has the structure:

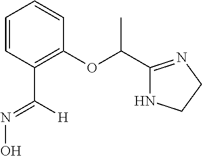 [SMILES: CC(Oc1ccccc1/C=N/O)C1=NCCN1]

and topically acceptable salts thereof.
23. The method according to claim 14, wherein the compound of formula (I) is capable of modulating adrenergic receptors of subtype α1A, α2A, and/or β2.
24. The method according to claim 23, wherein said compound is an adrenergic receptor agonist.
25. The method according to claim 23, wherein said compound is an agonist of α1A and/or α2A adrenergic receptors.
26. The method according to claim 23, wherein said compound has insubstantial agonist activity against β2 adrenergic receptor.
27. The method according to claim 14, wherein said composition is applied at least once daily for a period of at least four weeks.
28. A method for improving the appearance of human skin comprising topically applying to an area of the skin in need thereof a composition comprising, in a cosmetically acceptable vehicle, an effective amount of a compound having the structure of formula (I):

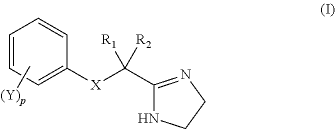

wherein X is selected from oxygen, sulfur, NR*, or CR*R*;
R₁ and R₂ are independently selected from hydrogen or R*, wherein R₁ and R₂ may together form a 3-6-membered ring;
Y is selected independently at each occurrence from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof;
p is an integer from 1-5;
R* is independently at each occurrence hydrogen or a straight chained, branched, or cyclic C1-C20 hydrocarbon radical, optionally substituted with one or more groups Y or with 1-6 heteroatoms selected from nitrogen, oxygen, sulfur, or halogen and combinations thereof; and topically acceptable salts thereof.
29. The method according to claim 28, wherein X is oxygen.
30. The method according to claim 28, wherein R₁ is methyl.
31. The method according to claim 28, wherein the compound has the structure of formula (II):

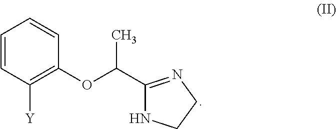

32. The method according to claim 28, wherein the compound of formula (I) is capable of modulating adrenergic receptors of subtype α1A, α2A, and/or β2.
33. The method according to claim 28, wherein the compound is selected from the group consisting of:

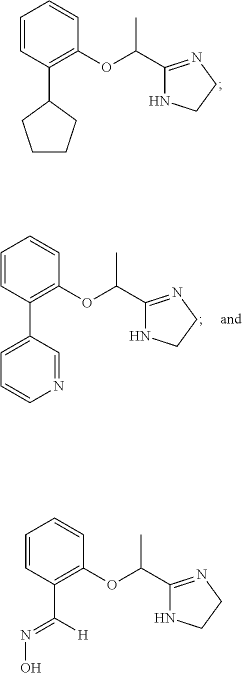 [SMILES: CC(Oc1ccccc1C1CCCC1)C1=NCCN1 ; CC(Oc1ccccc1-c1cccnc1)C1=NCCN1 ; CC(Oc1ccccc1/C=N/O)C1=NCCN1]

and topically acceptable salts thereof.
34. The method according to claim 33, wherein the compound has the structure:

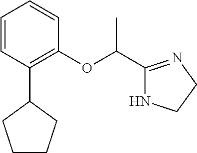 [SMILES: CC(Oc1ccccc1C1CCCC1)C1=NCCN1]

and topically acceptable salts thereof.
35. The method according to claim 33, wherein the compound has the structure:

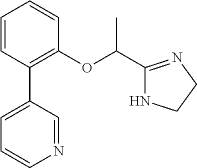 [SMILES: CC(Oc1ccccc1-c1cccnc1)C1=NCCN1]

and topically acceptable salts thereof.
36. The method according to claim 33, wherein the compound has the structure:

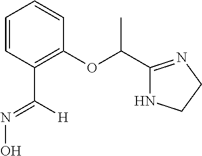 [SMILES: CC(Oc1ccccc1/C=N/O)C1=NCCN1]

and topically acceptable salts thereof.
37. The method according to claim 28, wherein the compound of formula (I) is capable of modulating adrenergic receptors of subtype α1A, α2A, and/or β2.
38. The method according to claim 37, wherein said compound is an adrenergic receptor agonist.
39. The method according to claim 37, wherein said compound is an agonist of α1A and/or α2A adrenergic receptors.
40. The method according to claim 37, wherein said compound has insubstantial agonist activity against β2 adrenergic receptor.
41. The method according to claim 28, wherein improving the appearance of human skin comprises improving skin tone and/or reducing dark circles under the eye area.
42. The method according to claim 28, wherein the improvement in the appearance of human skin is selected from a group consisting of:

(a) treatment, reduction, and/or prevention of fine lines or wrinkles;

(b) reduction of skin pore size;

(c) improvement in skin thickness, plumpness, and/or tautness;

(d) improvement in skin smoothness, suppleness and/or softness;

(e) improvement in skin tone, radiance, and/or clarity;

(f) improvement in procollagen, and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization;

(o) increase in skin elasticity and/or resiliency;

(p) treatment, reduction, and/or prevention of skin sagging;

(q) improvement in skin firmness; and

(r) reduction of pigment spots and/or mottled skin; and

(s) improvement of optical properties of skin by light diffraction or reflection.

What is claimed is:

1. A topical composition comprising, in a topically acceptable vehicle, an effective amount of a compound capable of modulation of adrenergic receptors having the structure of formula (I): 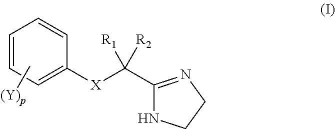
wherein X is selected from oxygen, sulfur, NR*, or CR*R*;
R₁ and R₂ are independently selected from hydrogen or R*, wherein R₁ and R₂ may together form a 3-6-membered ring;
Y is selected independently at each occurrence from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof;
p is an integer from 1-5;
R* is independently at each occurrence hydrogen or a straight chained, branched, or cyclic C₁-C₂₀ hydrocarbon radical, optionally substituted with one or more groups Y or with 1-6 heteroatoms selected from nitrogen, oxygen, sulfur, or halogen and combinations thereof;
and topically acceptable salts thereof.

2. The topical composition of claim 1, wherein X is oxygen.

3. The topical composition of claim 1, wherein R₁ is methyl.

4. The topical composition of claim 1, wherein the compound has the structure of formula (II):

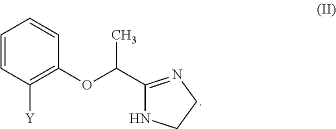

5. The topical composition of claim 1, wherein the compound is selected from the group consisting of: 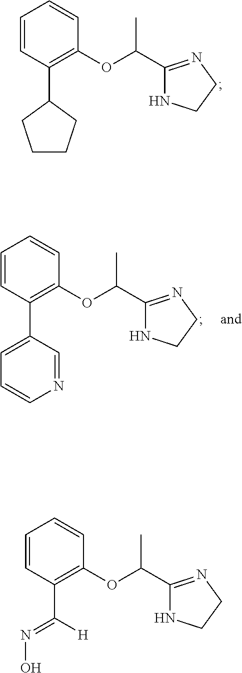 [SMILES: CC(Oc1ccccc1C1CCCC1)C1=NCCN1 ; CC(Oc1ccccc1-c1cccnc1)C1=NCCN1 ; CC(Oc1ccccc1/C=N/O)C1=NCCN1]
and topically acceptable salts thereof.

6. The topical composition according to claim 5, wherein the compound has the structure: 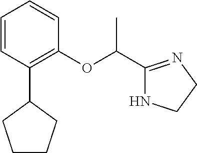 [SMILES: CC(Oc1ccccc1C1CCCC1)C1=NCCN1]
and topically acceptable salts thereof.

7. The topical composition according to claim 5, wherein the compound has the structure: 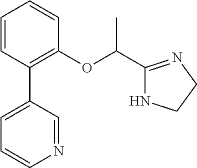 [SMILES: CC(Oc1ccccc1-c1cccnc1)C1=NCCN1]
and topically acceptable salts thereof.

8. The topical composition according to claim 5, wherein the compound has the structure: 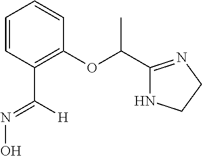 [SMILES: CC(Oc1ccccc1/C=N/O)C1=NCCN1]
and topically acceptable salts thereof.

9. The topical composition of claim 1, wherein the compound of formula (I) is capable of modulating adrenergic receptors of subtype α1A, α2A, and/or β2.

10. The topical composition of claim 9, wherein said compound is an adrenergic receptor agonist.

11. The topical composition of claim 9, wherein said compound is an agonist of α1A and/or α2A adrenergic receptors.

12. The topical composition of claim 9, wherein said compound has insubstantial agonist activity against β2 adrenergic receptor.

13. The composition according to claim 1, wherein said cosmetically acceptable vehicle comprises a water-in-oil, oil-in-water, silicone-in-water, or water-in-silicone emulsion and further comprises an emulsifier.

14. A method for modulating adrenergic receptors in human skin comprising topically applying to an area of the skin in need thereof a composition comprising, in a cosmetically acceptable vehicle, an effective amount of a compound having the structure of formula (I): 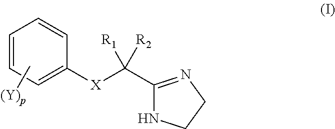
wherein X is selected from oxygen, sulfur, NR*, or CR*R*;
R₁ and R₂ are independently selected from hydrogen or R*, wherein R₁ and R₂ may together form a 3-6-membered ring;
Y is selected independently at each occurrence from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —CH(═N—OH); —SH; —SR*; —CN; —NC; —(C═O)—R*; —CHO; —CO₂H; —CO₂—; —CO₂R*; —(C═O)—S—R*; —O—(C=O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₅-C₈ aromatic hydrocarbon radical; or a C₅-C₈ heteroaryl radical, or combinations thereof;
p is an integer from 1-5;
R* is independently at each occurrence hydrogen or a straight chained, branched, or cyclic C₁-C₂₀ hydrocarbon radical, optionally substituted with one or more groups Y or with 1-6 heteroatoms selected from nitrogen, oxygen, sulfur, or halogen and combinations thereof;
and topically acceptable salts thereof.

15. The method according to claim 14, wherein said composition is applied at least once daily for a period of at least four weeks.

16. The method of claim 14, wherein the modulation of adrenergic receptors in human skin cells results in an improvement in appearance of the human skin containing the adrenergic receptors.

17. The method according to claim 16, wherein said composition is applied at least once daily for a period of at least four weeks.

18. The method according to claim 16, wherein the improvement of the appearance of human skin comprises improving skin tone and/or reducing dark circles under the eye area.

19. The method according to claim 16, wherein the improvement in the appearance of human skin is selected from a group consisting of:
(a) treatment, reduction, and/or prevention of fine lines or wrinkles;
(b) reduction of skin pore size;
(c) improvement in skin thickness, plumpness, and/or tautness;
(d) improvement in skin smoothness, suppleness and/or softness;
(e) improvement in skin tone, radiance, and/or clarity;
(f) improvement in procollagen, and/or collagen production;
(g) improvement in maintenance and remodeling of elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) improvement of skin appearance decreased by aging and/or menopause;
(n) improvement in skin moisturization;
(o) increase in skin elasticity and/or resiliency;
(p) treatment, reduction, and/or prevention of skin sagging;
(q) improvement in skin firmness; and
(r) reduction of pigment spots and/or mottled skin; and
(s) improvement of optical properties of skin by light diffraction or reflection.
